(19) United States
(12) Patent Application Publication
(10) Pub. No.: US 2016/0201009 A1
(43) Pub. Date: Jul. 14, 2016

(54) INTEGRATED OIL EXTRACTION APPARATUS

(71) Applicant: Aurelio Lopez, Antioch, CA (US)

(72) Inventor: Aurelio Lopez, Antioch, CA (US)

(21) Appl. No.: 14/594,089

(22) Filed: Jan. 10, 2015

(51) Int. Cl. C11B 9/02; B01D 11/02; A61K 36/752; A61K 36/53; A61K 36/63; C11B 1/10; A61K 36/185

(52) U.S. Cl. CPC C11B 9/025; C11B 1/108; B01D 11/0288; B01D 11/0207; A61K 36/185; A61K 36/53; A61K 36/63; A61K 36/752

(57) ABSTRACT

The invention provides a method for extracting oil from a starting plant material. The method includes passing a solvent through the plant material to obtain a first extract; allowing the first extract to reside in a pre-set temperature bath for a predetermined time to precipitate the impurities; filtering the precipitated impurities to obtain a second extract; and separating the solvent from the second extract to obtain a pure oil. An integrated oil extraction apparatus is provided. The apparatus includes an extraction chamber having an upper end and a lower end, a collection chamber and a de-wax chamber sandwiched between the extraction chamber and the collection chamber.

### FIELD OF THE INVENTION

The invention generally relates to the field of methods of chemical purification and particularly to an apparatus and method for integrated oil extraction.

### BACKGROUND OF THE INVENTION

Extraction of oils and other metabolically active compounds from plant materials basically involves two mechanisms that are mechanical or chemical. Mechanical methods include but are not limited to pressing, water extraction and dry ice extraction. Chemical methods, on the other hand, employ a solvent that dissolves the desired compounds in order to separate them from the plant material. The solvent used for extraction includes but is not limited to petroleum ether, methanol, ethanol, isopropyl alcohol, butane, hexane and carbon dioxide.

Ethanol in various concentrations has been used to extract active substances from the plant material. One significant disadvantage of ethanol is that it also extracts a considerable amount of plant pigments such as chlorophyll and carotenoids. Hexane and other petroleum-based solvents have good solvent activity. One significant disadvantage of using hexane as a solvent is risk of fire and explosion. CO₂ (carbon dioxide) is another solvent that is used commonly for extraction. Some advantages of using CO₂ are low environmental impact and nonexistent toxicity. However, CO₂ extraction systems are considerably more expensive. Butane is the solvent of choice for extraction. Butane is a non-polar solvent and thus does not carry an electrical charge in its molecules. Other advantages are low cost, easy availability and easy removal from the finished product.

Extraction of active substances from plant material using butane as a solvent can be achieved either in an open loop system or in a closed loop system. In an open loop system, extraction is done by blasting the plant material packed in a tube with butane. The extract along with the solvent is then collected in a collection bowl. Butane is then separated from the extract by evaporating the butane in the open air around the extractor. This results in the person performing the extraction sitting in a cloud of butane vapor, thus posing a serious health and safety hazard. Another disadvantage of an open loop system is that the butane evaporated cannot be recovered.

In a closed loop system, there is no gap through which the solvent can escape. The advantages of closed loop system are that as butane is not allowed to escape in the environment, it does not pose any health and safety hazard. Moreover, the solvent usage is drastically reduced as the solvent is re-circulated back in the system.

In addition to extraction, de-waxing is done to obtain pure extract. De-waxing is done to remove impurities. De-waxing is normally done as a separate process by passing the extract through a filter. This exposes the person to hazardous butane fumes. Additionally, de-waxing as a separate step requires additional set up and storage facilities.

The systems available in the art do not allow for removal of the waxes and other impurities in a single step. Thus, there is need for a system that not only allows for more effective and safer extraction but also provides impurity and toxin free extract in a single step.

### BRIEF DESCRIPTION OF THE DRAWINGS

So that the manner in which the recited features of the invention can be understood in detail, some of the embodiments are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a side view of the integrated oil extraction apparatus, according to an embodiment of the invention;

FIG. 2 shows an extraction chamber of the integrated oil extraction apparatus, according to an embodiment of the invention;

FIG. 3 shows a de-wax chamber of the integrated oil extraction apparatus, according to an embodiment of the invention;

FIG. 4 shows a side view of a collection chamber of the integrated oil extraction apparatus, according to an embodiment of the invention; and

FIG. 5 shows a purification apparatus downstream of the de-wax chamber, according to an embodiment of the invention.

### SUMMARY OF THE INVENTION

One aspect of the invention provides a method for integrated oil extraction from a starting plant material. The method includes passing a solvent through the plant material to obtain a first extract. The first extract is allowed to reside in a pre-set temperature bath for a predetermined time to precipitate the impurities. The precipitated impurities are then filtered to obtain a second extract. The solvent is separated from the second extract to obtain a pure de-waxed oil.

Another aspect of the invention provides an integrated apparatus for extraction of active compounds from the plant material. The apparatus comprises of an extraction chamber having an upper end and a lower end, a collection chamber and a de-wax chamber sandwiched between the extraction chamber and the collection chamber.

### DETAILED DESCRIPTION OF THE PREFERRED EMBODIMENT

Various aspects and embodiments of the invention provide a method and an apparatus for extraction of active compounds from the plant material. The method comprises of passing a solvent through the plant material to obtain a first extract, allowing the first extract to reside in a pre-set temperature bath for a predetermined time to precipitate the impurities, filtering the precipitated impurities to obtain a second extract and separating the solvent from the second extract to obtain a pure de-waxed oil. The apparatus comprises of an extraction chamber having an upper end and a lower end, a collection chamber and a de-wax chamber sandwiched between the extraction chamber and the collection chamber.

The extraction process is carried out using butane as a solvent. Butane is a gas at room temperature and atmospheric pressure. Butane is a highly inflammable, colorless and easily liquefiable gas. Butane generates vapor pressure of approximately 20 psi at 75° F. (24° C.) and has a boiling point of around −1° C. The plant material used for extraction includes but is not limited to cannabis, hops, orange peel, lavender, jasmine and other oil containing herbs and medicinal plants. In one embodiment of the invention, cannabis is selected as the plant material. The plant material can be leaves, flowers, florets, roots, bark or any other plant part. The plant material is dried and powdered. A fine bed of powdered plant material is prepared by pounding and pressing the plant material. Butane is sprayed on the plant material for a predetermined time period. As the butane comes in contact with the plant material, all active compounds are dissolved in the butane. The solution containing the dissolved active compounds in butane is then sent to a de-waxing step. The solution is allowed to reside in a pre-cooled chamber for about 3-4 hrs. Pre-cooling is typically done by using dry ice. Low temperature results in precipitation of the impurities, which are extracted from the plant material along with the active compounds. The impurities include but are not limited to waxes, lipids, terpenes, gums and pigments. Subsequent to precipitation of impurities, the solution is passed through a filtration process. During filtration, the precipitated impurities are removed. The filtrate is then collected. The collected filtrate is then subjected to the solvent recovery step.

Solvent recovery is achieved by converting the solvent from liquid phase to vapor phase. The solvent in vapor phase is again converted to liquid phase in a separate container, which can be used again for extraction of a fresh batch of plant material. After solvent recovery, a pure de-waxed oil is obtained. The de-waxed oil includes but is not limited to an herbal oil, an essential oil and a medicinal oil.

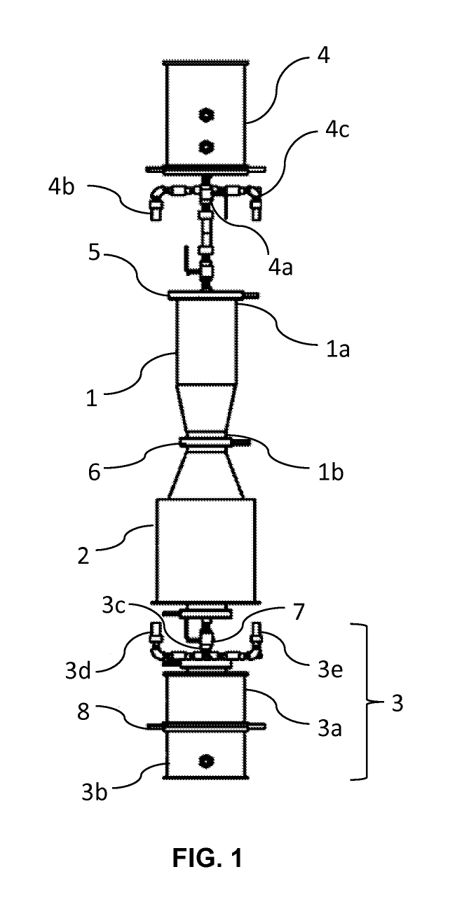

FIGS. 1-5 generally describe the components and arrangement of the integrated oil extraction apparatus. FIG. 1 particularly shows a side view of the integrated oil extraction apparatus, according to an embodiment of the invention. The integrated oil extraction apparatus includes an extraction chamber 1, a de-wax chamber 2, a collection chamber 3 and a solvent reservoir 4. The extraction chamber 1 includes an upper end 1a and a lower end 1b. The extraction chamber has a clamp 5 for securing a cap (not shown in figure). The lower end 1b of the extraction chamber 1 is attached to the de-wax chamber 2 with the help of a clamp 6. A gasket is provided between the extraction chamber 1 and the de-wax chamber 2 to ensure maximum compression and prevent the leakage of solvent. The length of the extraction chamber 1 can be altered depending on the quantity of the plant material. The de-wax chamber 2 is in turn connected to a collection chamber 3 through an adapter 7. The collection chamber 3 includes an upper collection chamber 3a and a lower collection chamber 3b. The upper collection chamber 3a is attached to a lower collection chamber 3b by the means of a clamp 8. The collection chamber 3 is provided with a first valve 3c for receiving fluid from the de-wax chamber 2. The first valve 3c is unidirectional. A second valve 3d is provided for passing the recovered solvent back to the solvent reservoir 4 at the end of the purification. A third valve 3e is also provided for attaching a vacuum pump.

The solvent reservoir 4 is connected to the upper end 1a of the extraction chamber 1. The solvent reservoir 4 is provided with a first valve 4a for injecting the solvent into the extraction chamber 1. The first valve 4a is unidirectional. A second valve 4b is provided for receiving the recovered solvent at the end of the purification. The second valve 4b is unidirectional. Additionally, a third valve 4c is provided. In an example of the invention, the third valve 4c can be used for connecting a pressure gauge.

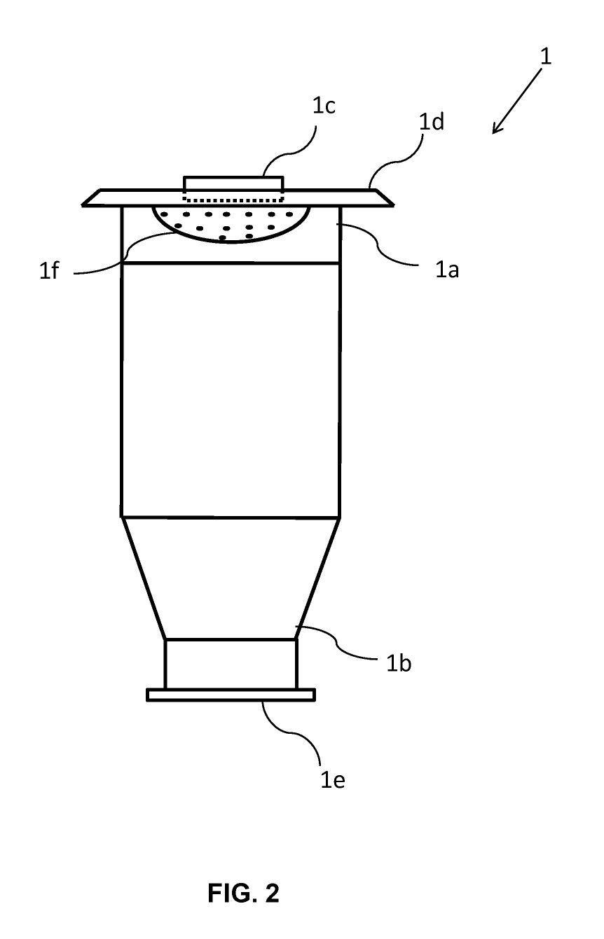

FIG. 2 shows an extraction chamber 1 of the integrated oil extraction apparatus, according to an embodiment of the invention. The extraction chamber 1 includes an inlet 1c, an extractor cap 1d and an outlet 1e. The extractor cap 1d is fitted with a spray ball 1f for evenly distributing the solvent. The length of the extraction chamber 1 can be altered based on the amount of material that is to be processed. The extraction chamber 1 is made air tight through a gasket or a seal. The gasket ensures maximum compression and prevents the escape of solvent. The extraction chamber is made of a non-corroding material. Additionally, the inner wall of the extraction chamber 1 can be coated with food grade paint.

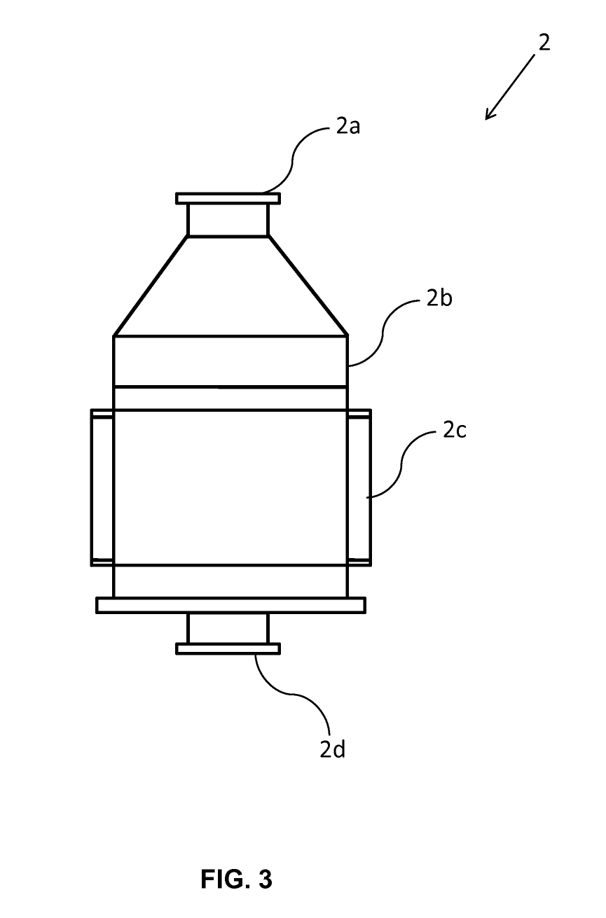

FIG. 3 shows a de-wax chamber 2 of the integrated oil extraction apparatus, according to an embodiment of the invention. The de-wax chamber 2 includes an inlet 2a, an inner chamber 2b, a sleeve 2c and an outlet 2d. The sleeve 2c is provided for facilitating a cold water bath. The de-wax chamber 2 is made of a non-corroding material.

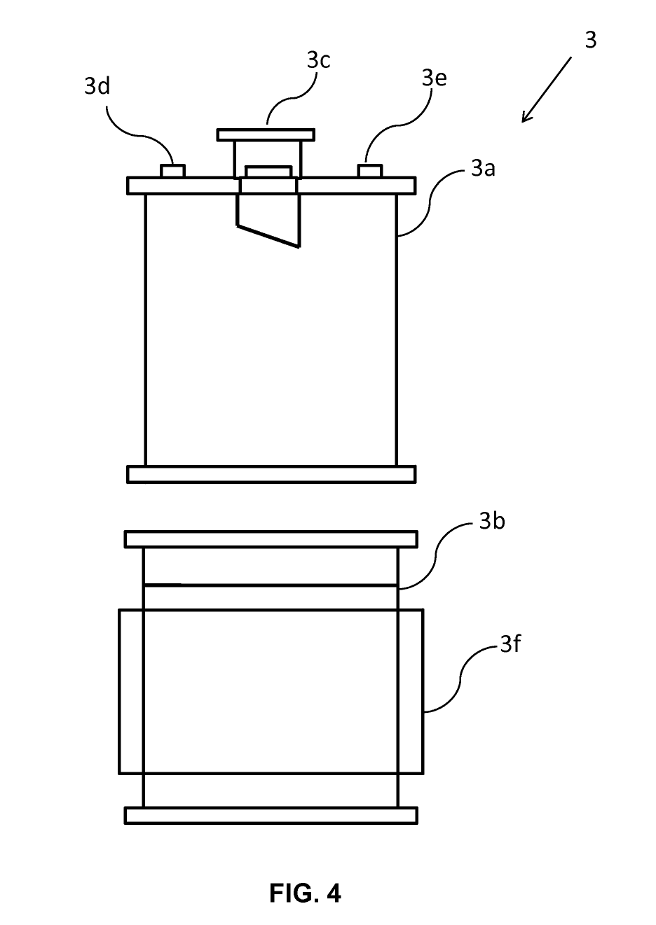

FIG. 4 shows a side view of a collection chamber 3 of the integrated oil extraction apparatus, according to an embodiment of the invention. The collection chamber 3 includes an upper collection chamber 3a and a lower collection chamber 3b. The upper collection chamber 3a is attached to the lower collection chamber 3b by using a clamp 8 (not shown in FIG. 4). The removal of clamp 8 facilitates easy recovery of oil from the lower collection chamber 3b. A gasket is provided between the upper collection chamber 3a and the lower collection chamber 3b, to ensure maximum compression and prevent solvent leakage. The lower collection chamber 3b is fitted with a sleeve 3f for facilitating a hot/cold water bath. The collection chamber 3 is provided with a first valve 3c for receiving fluid from the de-wax chamber 2. The first valve 3c is unidirectional. A second valve 3d is provided for passing the recovered solvent back to the solvent reservoir 4 at the end of the purification. A third valve 3e is also provided for attaching a vacuum pump.

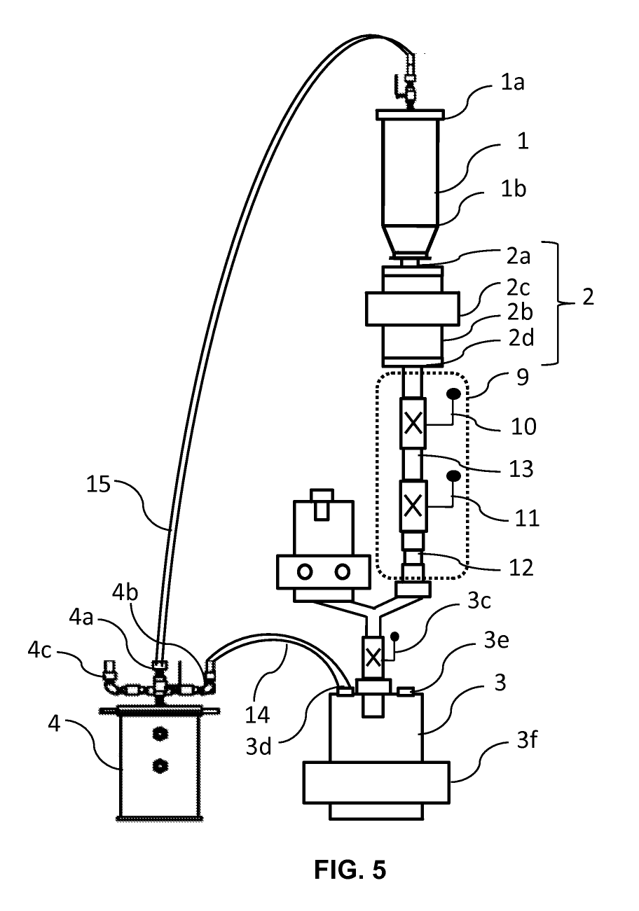

FIG. 5 shows a purification apparatus downstream of the de-wax chamber, according to an embodiment of the invention. The purification apparatus 9 is sandwiched between the de-wax chamber 2 and the collection chamber 3. The purification apparatus 9 includes a plurality of valves 10 and 11, a filter 12 and a visual indicator 13. The valves 10 and 11 are used for controlling the flow of fluid from the de-wax chamber 2 to the filter 12. The flow of fluid from the filter 12 to the collection chamber 3 is controlled by the valve 3c. The visual indicator 13 is placed between the valves 10 and 11. The visual indicator 13 described herein is a process observation equipment. In one example of the invention, the visual indicator 13 is a sight glass. A recovery line 14 is connected between the valve 3d of the collection chamber 3 and the valve 4b of the solvent reservoir 4. The recovery line 14 described herein includes but is not limited to a pipe, a tube, a hose or any other channel capable of transmitting solvent. A fill line 15 is connected between the valve 4a of the solvent reservoir 4 and the extraction chamber 1. The fill line 15 described herein includes but is not limited to a pipe, a tube, a hose or any other channel capable of transmitting solvent. Further, the recovery line 14 and fill line 15 are made up of a non-reacting material.

A fine bed of powdered plant material is prepared by pounding and pressing the plant material in the extraction chamber 1. The plant material is filled tightly so as to remove any air pockets. Presence of air pockets slows down the extraction process. After filling the plant material in the extraction chamber 1, the extractor cap 1d is placed and the extraction chamber is clamped to prevent leakage of the solvent. A pressure of about 30 psi is applied to the apparatus. The pressure is achieved by connecting the collection chamber 3 to the vacuum pump. The apparatus is then cooled to a temperature of about −70° C. to about −80° C. The temperature is achieved by putting the lower collection chamber 3b in ice water bath. Alternatively, the temperature can be achieved by filling dry ice in the sleeve 3f provided outside the lower collection chamber 3b. Dry ice is also filled in the sleeve 2c provided outside the de-wax chamber 2.

The solvent in the solvent reservoir 4 is activated by keeping the solvent reservoir 4 in a hot water bath for about 5 to 10 minutes. High temperature converts the solvent from the liquid phase to the vapor phase. The solvent reservoir 4 is then connected to the extraction chamber 1 through the valve 4a. The valve 4a allows the solvent in the vapor phase to be purged through the inlet 1c of the extraction chamber 1. As soon as the solvent comes in contact with the pre-cooled extraction chamber 1, the solvent in the vapor phase is converted into the liquid phase. The spray ball if provided on the extractor cap 1d sprays the solvent on the plant material. The solvent is sprayed on the plant material for a predetermined time period. As the solvent comes in contact with the plant material, all active compounds are dissolved in the solvent.

The solution containing the active compounds is then sent to the de-wax chamber 2, where the solution is allowed to reside in for about 3-4 hrs. The low temperature of the de-wax chamber 2 results in precipitation of the impurities that are extracted from the plant material along with the active compounds. Subsequent to precipitation of the impurities, the valves 10 and 11 are opened and the solution is passed through the filter 12. The passing of solution is visible through the sight glass 13. The precipitates are removed by the filter 12 and the filtered solution is collected in the collection chamber 3.

Once all the filtered solvent is collected in the collection chamber 3, the inlet valve 3c is closed. The filtered solvent is then subjected to a solvent recovery process, where the solvent is evaporated and pure de-waxed oil is obtained. For solvent recovery, the valve 3d of the collection chamber 3 is connected to the valve 4b of the solvent reservoir 4 via the recovery line 14. The collection chamber 3 is then put in hot water bath. Alternatively, hot water can be filled in the sleeve 3f provided outside the lower collection chamber 3b. Simultaneously, the solvent reservoir 4 is placed in ice water bath. This creates a temperature gradient from the collection chamber 3 to the solvent reservoir 4. Additionally, the recovery process can be assisted by connecting the solvent reservoir 4 to the vacuum pump. The high temperature in the collection chamber 3 converts the solvent in the liquid phase to the vapor phase. Due to temperature and pressure gradient, the solvent in the vapor phase passes to the solvent reservoir 4. In the solvent reservoir 4, the solvent in the vapor phase is again converted to the liquid phase due to low temperature. Once all the solvent evaporates and is collected back in the solvent reservoir 4, only pure oil containing active compounds is left in the collection chamber 3. The collection chamber 3 is then opened by removing the clamp 8 and pure de-waxed oil is scraped out.

While preferred embodiments of the present invention have been shown and described herein, it will be obvious to those skilled in the art that such embodiments are provided by way of example only. Numerous variations, changes, and substitutions may now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for extracting oil from a starting plant material, the method comprising;
passing a solvent through the plant material to obtain a first extract;
allowing the first extract to reside in a pre-set temperature bath for a predetermined time to precipitate impurities from said first extract;
filtering the precipitated impurities to obtain a second extract; and
separating the solvent from the second extract to obtain a pure oil, the separating step further comprising:
converting the solvent from a liquid phase to a vapor phase;
collecting the solvent in the vapor phase along a temperature gradient; and
reconverting the recovered solvent from the vapor phase to the liquid phase.

2. The method of claim 1, wherein the oil is selected from the group consisting of an herbal oil, an essential oil and a medicinal oil.

3. The method of claim 1, wherein the plant material is selected from the group consisting of cannabis, hops, orange peel, lavender, and jasmine.

4. The method of claim 1, wherein the solvent is passed through the plant material in liquid phase.

5. The method of claim 1, wherein the solvent is butane.

6. The method of claim 1, wherein the temperature bath is prepared by packing ice or dry ice.

7. The method of claim 1, wherein the temperature of the bath is maintained at a temperature ranging from about −70° C. to about −80° C.

8. The method of claim 1, wherein the impurities comprise waxes, lipids, terpenes, gums and pigments.

9. The method of claim 1, wherein the temperature gradient enables liquid-vapor-liquid transition of the solvent.

10. The method of claim 1, wherein the method is completed within a closed system.

11. An integrated oil extraction apparatus, the apparatus comprising:
an extraction chamber having an upper end and a lower end, the upper end connected via a connection to a solvent reservoir;
a collection chamber; and
a de-wax chamber sandwiched between the extraction chamber and the
collection chamber, and connected to the lower end of the extraction chamber.

12. The apparatus of claim 11, wherein the solvent reservoir is provided with a first valve for injecting the solvent into the extraction chamber.

13. The apparatus of claim 12, wherein the extraction chamber is provided with a spraying mechanism for solvent infusion.

14. The apparatus of claim 11, wherein said connection is unidirectional.

15. The apparatus of claim 11, wherein the de-wax chamber is provided with at least one filter for de-waxing the extract obtained from the extraction chamber.

16. The apparatus of claim 11, wherein the de-wax chamber is provided with a sleeve for facilitating a cold water bath.

17. The apparatus of claim 11, wherein the collection chamber comprises of an upper collection chamber coupled to a lower collection chamber.

18. The apparatus of claim 11, wherein the collection chamber is provided with a sleeve for facilitating a hot water bath and a cold water bath.

19. The apparatus of claim 11, further wherein the collection chamber is configured for recovery of the solvent.

20. The apparatus of claim 11, wherein the collection chamber is connected to a second valve for transferring the recovered solvent to the solvent reservoir.
